(19) United States
(12) Patent Application Publication
(10) Pub. No.: US 2024/0000669 A1
(43) Pub. Date: Jan. 4, 2024

(54) NOVEL ENDODONTIC IRRIGANT

(71) Applicant: INTER-MED, INC., Racine, WI (US)

(72) Inventors: John Baeten, Racine, WI (US); Thomas KANDATHIL, Racine, WI (US); Alex JOHNSON, Racine, WI (US); Katherine BARRY, Racine, WI (US)

(21) Appl. No.: 18/250,642

(22) PCT Filed: Oct. 26, 2021
(86) PCT No.: PCT/US2021/072043

§ 371 Date: Apr. 26, 2023

Related U.S. Application Data
(60) Provisional application No. 63/105,561, filed on Oct. 26, 2020.

(51) Int. Cl. A61K 6/52

(52) U.S. Cl. CPC A61K 6/52

(57) ABSTRACT

A medicament for dental procedures includes a first component comprising an oxidizing agent, and a second component comprising a calcium complexing agent, wherein when the first component is mixed with the second component, the pH is greater than 6.2. Kits including the medicament and methods of use of the medicament are disclosed.

### CROSS-REFERENCE TO RELATED APPLICATIONS

This application is a National Stage application of International Application No. PCT/US2021/072043 filed Oct. 26, 2021, which claims the benefit of U.S. Provisional Application No. 63/105,561, filed Oct. 26, 2020, which are incorporated by reference herein in their entirety.

### BACKGROUND

The inner portion of a tooth includes a pulp cavity that contains soft living tissue, or the “pulp,” of the tooth. The pulp includes connective tissue, blood vessels, other living cells, and nerve endings. The pulp cavity includes an upper pulp chamber and root canals that extend to the apex or apical section of the tooth deeper into the jaw. The outer (visible) portion of the tooth is referred to as the crown and has a covering of enamel. The hard enamel protects softer dentinal tissues in the upper portion of the tooth. The enamel includes, or consists of, a hard, calcium-based substance, hydroxyapatite. The dentin tissue contains a matrix of minute tubules interspersed with collagen fibers that surround and protect the tooth pulp. The outer (non-visible) portion of the tooth root is covered with cementum, a thin hard tissue that joins the root to the surrounding bone through Sharpey's fibers. Dental decay, or caries, is caused by bacteria accumulating on teeth and forming a biofilm (plaque). The biofilm produces acids that dissolve and weaken the hydroxyapatite of the tooth, thereby causing decay.

Various chemical and physical irritants can cause irritation and even necrosis of the pulp. The most common causes for pulpal inflammation (pulpitis) are bacteria and/or their products entering the pulp through a deep carious lesion or a leaking cavity filling (also called dental restoration). In such a case, an inflammatory reaction in the pulp can start long before bacteria invade the pulp tissue. The inflammatory reaction is first initiated by bacterial antigens interacting with the local immune system. As long as the carious lesion has not entered the pulp, the pulpal inflammation is likely to be reversible. When the carious lesion does reach the pulp and the hard tissue barrier is breached, however, bacteria can invade the pulp. Even after this point, the infection may remain relatively superficial, and most of the pulp tissue remains vital and bacteria free. For this reason, one approach for an endodontic treatment of pulpitis may be treatment of inflammation and prevention of an infection.

In apical periodontitis, bacteria invade further and colonize the entire root canal system. Apical periodontitis is an inflammatory process in the periradicular tissues caused by microorganisms in the necrotic root canal. Accordingly, to promote healing of apical periodontitis, microorganisms within the root canal system must be eliminated. Provided that the dental disease is not too progressed, dental professionals will use root canal treatment procedures to remove the infected tissue from the tooth and replace it with an inert, biocompatible material.

The root canal system of a tooth is complex, and many treatment methods can be used depending upon the condition of the patient and approach of the practitioner. Typically, endodontic procedures involve three steps. The first step is called instrumentation and entails opening the tooth and widening the root canal system utilizing a series of semi-rigid metal endodontic files, often times in conjunction with endodontic irrigants (e.g. sodium hypochlorite) to reduce the debris level created by the endodontic files. This step removes some organic and inorganic material, but its primary goal is to enlarge the root canal(s) to allow for the introduction of small cannula used during the second step, call irrigation. Irrigation entails flushing of the root canal system using small cannula (called irrigation needles) attached to syringes of various aqueous-based endodontic irrigants, including sodium hypochlorite. The goal of the irrigation step is to debride the root canal system and solubilize any remaining debris and smear layer introduced during instrumentation. The third and final step is called obturation, wherein an elastic (rubbery) material called gutta percha is used to fill the root canal system. After the endodontic procedure is finalized, a standard dental restorative procedure (i.e. composite filling or crown) is used to complete the structure and aesthetics of the tooth.

Due to the complexities of the root canal system, it is known in the dental literature that the first step (instrumentation with endodontic files) leaves a significant portion of the root canal system untouched. To elaborate, the sequential use of endodontic files leaves a smear layer of cut organic and inorganic debris on the dentin walls that can only be removed through additional flushing and irrigation using endodontic irrigants. Additionally, the design of semi-rigid metal files means they are physically limited in their ability to access all areas of the root canal system. For example, a 2010 micro-CT study confirmed that endodontic files used during instrumentation only physically contact 21.1%-40.4% of the canal area, leaving a significant percentage uncleaned. This is especially true for hard-to-reach canal anatomy, such as fins, isthmuses and lateral canals which cannot be adequately treated with semi-rigid endodontic files made of metal. As such, the irrigation stage of root canal therapy is arguably the most important since the aqueous based irrigant solutions can flow to and reach the areas left untouched during instrumentation.

Dentin, the “inner” dental hard tissue, contains a multitude of small channels, known as dentinal tubules. During mechanical instrumentation/treatment during root canal therapy, a smear layer is produced, which blocks these tubules and allows bacteria to hide and multiply in these hollow spaces. The term “smear layer” is known to those of skill in the art of dentistry and refers to the complex accumulation of soluble and insoluble organic and inorganic debris resulting from the mechanical preparation of a tooth surface. The smear layer includes cutting debris, tooth particles, microorganisms, necrotic material, and other substances resulting from preparation, and can include a superficial layer on the surface of a prepared tooth along with a layer or layers that are packed into the adjacent dentinal tubules at varying depths.

The smear layer, which consists of organic (pulp tissue, bacterium, collagen and other fibrous proteins, etc.) and inorganic (dentine chips and nonspecific inorganic contaminants) components, may be removed in order to facilitate the antimicrobial action of antiseptic agents. Removal of the smear layer is further necessary to allow optimal bonding of restorative or root-filling materials to dentin. In order to remove the smear layer created during instrumentation, and reach areas of the root canal system untouched by instrumentation, a medicament or combination of medicaments may be used. Typically these medicaments are called irrigants, irrigating solutions, or endodontic irrigants, among other common synonymous terms. Irrigation plays the main role in proper cleaning and eradication of microbes from the root canal system.

A variety of medicaments and disinfectants for dental procedures as well as methods for removing smear layers, buildup of debris and bacteria formed during the preparation of tooth surfaces in procedures such as root canal treatment, restoration, and the like, have been developed. Chelating agents were introduced into endodontics as an aid for the preparation of narrow and calcified root canals, where a liquid solution of ethylenediaminetetraacetic acid (EDTA) or citric acid were thought to chemically soften the root canal dentine and dissolve the smear layer as well as to increase dentine permeability. Current chemical agents to remove a smear layer therefore often include EDTA and organic acids such as citric acid in their formulations. EDTA is frequently used in aqueous gels for root canal therapy because its chelating capacity may reduce stress on nickel-titanium instruments used in the mechanical treatment/instrumentation of the root canal system. Where agents based on EDTA are capable of removing the inorganic portion of the smear layer, however, the organic portion of smear layer is often left intact in the root canal system.

A root canal treatment of an infected canal system is typically performed in two visits. Between these visits, a temporary filling material/disinfectant is placed in the root canal system which aims to destroy remaining microorganisms and prevent reinfection. Calcium hydroxide (Ca(OH)₂) is commonly used between treatment appointments due to its antibacterial effect, attributed at least in part to the release of hydroxide ions over time and their diffusion through dentine. Frequently, aqueous solutions of NaOCl are used to rinse root canals during and after mechanical debridement, while calcium hydroxide slurries are placed in the thus-partially cleaned root canal system to disinfect between two dental visits. Calcium hydroxide exerts its antibacterial effect in the root canal system as long as a high pH value is maintained. The use of Ca(OH)₂ placed as a disinfectant in the root canal system, however, has certain drawbacks. For example, calcium hydroxide has a low solubility in water and consequently the onset of its disinfecting effect is slow. Moreover, it has to be removed before the canal is filled with a restorative or root-filling material, which is cumbersome, since calcium hydroxide particles are hard to retrieve from the root canal system, given their complexity.

Locally used endodontic disinfectants, either irrigating solutions or interappointment medicaments, are effective against a wide spectrum of microorganisms. They affect a range of vital functions of the microbial cell, resulting rapidly in cell death. Hypochlorous acid interferes with oxidative phosphorylation and other membrane-associated functions of the cell as well as DNA synthesis inside the cell. Hypochlorite solution is effective against bacteria and yeast; even bacterial spores are killed with high concentration (5%) sodium hypochlorite. The antimicrobial effectiveness of the solution arises due to its ability to oxidize and hydrolyze cell proteins, and osmotically draw fluid from cells due to its hypertonicity. Hypochlorite also has the ability to dissolve organic debris (for example necrotic pulp tissue). However, hypochlorite cannot adequately remove smear layer by itself, leaving the inorganic components and organic components which are not reachable by the solution, that has formed on canal walls that have been in contact with rotary preparation instruments.

Sodium hypochlorite (NaOCl) is a very efficient agent to dissolve the organic smear layer components and a strong antimicrobial agent. EDTA and citric acid, however, strongly interact with oxidizing agents such as NaOCl and render the oxidizing agents ineffective by accelerating the degradation of the available hypochlorite anion OCl⁻.

Nevertheless, despite the longstanding history of endodontic therapy, there is uncertainty regarding the protocol for a successful root canal treatment, specifically in regard to the irrigation protocol. This uncertainty includes the concentrations of irrigating solutions, the volume of each medicament required, the sequence of medicaments, the necessary flow rate, the requirement to rinse between irrigants, and the necessity for medicament activation. While typical endodontic irrigation regiments include the sequential use of 2-6% NaOCl and 17% EDTA, to remove organic and inorganic debris, respectively, the high quality of endodontic dentistry may be impacted by the chosen method of processing the root canals of the tooth.

Methods for disinfecting and removing a smear layer from prepared tooth surfaces which involve irrigating the tooth surface with a disinfectant solution containing doxycycline or tetracycline, a surfactant such as polysorbate and an organic acid such as citric acid are known from WO 03/061506 A1, the entire contents of which are incorporated herein by reference.

US 2003/0156980 A1, the entire contents of which are incorporated herein by reference, discloses methods for disinfecting and cleaning dental root canals using a viscous sodium hypochlorite (NaOCl) composition, for example a disinfectant including an aqueous sodium hypochlorite solution and a gelling agent such as fumed silica or carboxypolymethylene.

WO 2005/123007, the entire contents of which are incorporated herein by reference, discloses a pharmaceutical preparation for use in a dental treatment including an aqueous solution of 1-hydroxyethane-1,1′-diphosphonate (HEDP) as calcium complexing agent and a diluted NaOCl solution as oxidizing agent. This disclosure yields a final product which, however, requires dissolving the HEDP powder in an aqueous solution of NaOCl, which can lead to incomplete mixing, poor solubility of HEDP and a relatively non-stable solution as evident by testing disclosed in this patent. Additionally, the composition described in this patent requires storage conditions that differ for the individual components leading to transportation and storage difficulties.

In one aspect, to obtain high quality treatment and reproducible results of treatment of the root canal system of the tooth, it may be desirable to use a simplified procedure consisting of a single irrigating solution that is able to provide the necessary tissue dissolution and smear layer removal. This may also be cost advantageous to a dental professional as only one syringe and irrigating tip would be required per procedure versus 3-4 syringes and irrigating tips. This may also save a dental professional time as only one irrigation solution may be needed for an endodontic procedure.

Disclosed herein is a composition, useful for irrigating prepared tooth surfaces, that includes an oxidizing agent for the solubilization of organic matter and disinfection, particularly an oxidizing agent capable of dissolving organic matter, including eradicating bacterium and fungi, and a calcium complexing agent for the removal of smear layer.

### SUMMARY

The present disclosure provides a medicament for use in dental treatments, particularly for use in root canal therapy in vivo.

In one aspect, the disclosure provides a composition useful for rinsing prepared tooth surfaces. The action of the composition is two-fold. The composition effectively removes buildup of undesirable debris (smear layer) formed during the preparation of tooth surfaces during dental procedures, including the solubilization and removal of organic-based and inorganic-based debris. The composition further disinfects the tooth surface, prepares the tooth for subsequent bonding agents to be applied by roughening the surface and exposing collagen fibers and, depending on the pH of the solution, may also solubilize organic matter (i.e. tissue dissolution).

In one aspect, the present application discloses a medicament that includes a first component comprising an oxidizing agent, and a second component comprising a metal complexing agent, particularly a calcium complexing agent. When the first component is mixed with the second component, the pH is greater than 6.2, and wherein the resulting pH of the combined solution determines whether the cleaning solution can effectively dissolve organic matter. For satisfactory tissue dissolution the pH of the medicament is preferably above 7.5, more preferably between 8 and 10, and most preferably between 9.25 and 10. The oxidizing agent may be a hypohalogen salt, which may be sodium hypochlorite (NaOCl). The calcium complexing agent may be a phosphonate, such as 2-phosphonobutane-1,2,4-tricarboxylic acid (PBTC), or a polyphosphate, such as sodium tripolyphosphate (STPP).

In another aspect, disclosed herein is a dispensing system containing the medicament. The dispensing system may include a first container for the first component, and a second container for the second component, such that the first and second components are separated from one another until a practitioner mixes them either at the point of use or just prior to the procedure. Suitable non-limiting examples of dispensing systems would include a dual cartridge syringe or a dual chamber bottle. In certain aspects of the invention, the dispensing system may comprise aspects of the delivery system, for example, a dual barrel syringe to which a tip is connected such that the medicament can be dispensed and delivered to the treatment site. Preferably, the at least two components are aqueous-based liquids for quick mixing and homogenization; however, it could be possible to have at least one component be a powder and the at least one other component be an aqueous-based liquid.

It should be understood that the medicament compositions and dispensing systems detailed herein can be split into more than two separate components which are then mixed together for clinical use. Therefore, although many of the examples described herein include two components, the disclosure should not be construed as limiting the compositions and systems to two components. Many modifications and adaptations to the compositions and systems of the disclosure are possible and apparent to one of ordinary skill in the art. For example, by modifying the compositions of the disclosure to include more than two components. Stated another way, the compositions disclosed herein should be understood to include at least two components.

In another aspect, methods of use of the medicament are disclosed. Such methods include methods of cleansing, disinfecting and dissolving inorganic and organic tooth debris for various dental treatments. These methods can be performed in vivo, ex vivo, or in a laboratory setting, for example, in relation to dental prosthetics. Typically, when used in vivo for endodontic procedures, the endodontic irrigants/medicaments are applied via a syringe attached to an appropriately sized needle (e.g. 27 gauge, 30 gauge, etc.). The needles maybe be blunt cut, skived, or side cut for the purpose of modulating irrigant delivery and flow.

In yet another aspect, processes for preparing the medicament compositions are disclosed.

### BRIEF DESCRIPTION OF THE DRAWINGS

The disclosure can be better understood with reference to the following drawings and description, in which:

FIG. 1 depicts a bottle capable of interlocking with itself to form a dual chamber dispensing system suitable for storage and delivery of the disclosed invention;

FIG. 2 depicts a dual cartridge syringe system capable of attaching to a mixing tip suitable for storage and delivery of the disclosed invention;

FIG. 3 depicts a mixing tip capable of affixing to the dual cartridge syringe of FIG. 2 that allows for adequate mixing of the individual components contained within the dual cartridge syringe prior to application at a treatment site;

FIG. 4 illustrates the mass loss (mean %) of tissue samples incubated in 30 mL of aqueous solutions of various agents after 30 minutes;

FIG. 5 illustrates chlorine speciation profile as a function of pH; and

FIG. 6 illustrates mass loss of tissue samples incubated in 30 mL of sodium hypochlorite at different pH values after 30 minutes.

FIG. 7 illustrates the tissue dissolution performance of Formula A (room temperature and 55° C.) compared to 3% NaOCl. Formula A shows improved tissue dissolution at room temperature compared to a NaOCl solution of similar concentration. Heating of Formula A further enhances its tissue dissolution ability.

FIG. 8 illustrates the stability of Formula A versus a co-mix solution of EDTA and 8% NaOCl. Formula A showed short term stability as NaOCl levels were greater than or equal to 2% at five hours.

FIG. 9 illustrates the antimicrobial efficacy of various irrigants against E. faecalis biofilm. Formula A was the only irrigant that was able to retain its clinical efficacy, in terms of antimicrobial activity, in the presence of dentin.

FIG. 10 illustrates the antimicrobial efficacy of various irrigants against E. faecalis biofilm in the presence or absence of smear layer. Formula A was more effective than the standard endodontic irrigation regiment (6% NaOCl for 5 min plus 17% EDTA for 1 min) in half the time.

FIG. 11 illustrates a zone of inhibition test, demonstrating that Formula A provided the greatest zone of inhibition as compared to other endodontic irrigants.

### DETAILED DESCRIPTION

The following paragraphs define in more detail the embodiments of the invention described herein. The following embodiments are not meant to limit the invention or narrow the scope thereof, as it will be readily apparent to one of ordinary skill in the art that suitable modifications and adaptations may be made without departing from the scope of the invention, embodiments, or specific aspects described herein. All patents and publications cited herein are incorporated by reference herein in their entirety.

For purposes of interpreting this specification, the following abbreviations, terms and definitions will apply and whenever appropriate, terms used in the singular will also include the plural and vice versa. In the event that any definition set forth below conflicts with any document incorporated herein by reference, the definition set forth below shall control.

The terms “room temperature” or “ambient temperature,” as used herein, refer to common ambient temperatures, for example, ranging from about 18° C. to about 27° C.

The term “treating” refers to administering a therapy in an amount, manner, or mode effective to improve a condition, symptom, or parameter associated with a disorder. In some aspects, treating refers to the treatment of a dental ailment such as an infected tooth.

As used herein, “a” or “an” means one or more than one, unless otherwise specified.

The transitional phrase “comprising,” which is synonymous with the terms “characterized by,” “include,” “including,” “contain,” “containing,” “has,” or “having,” and the like, is inclusive or open-ended and does not exclude additional, un-recited elements, components, ingredients and/or method steps.

The term “patient” or “subject” refers to mammals and humans. Thus, in some aspects, the subject is a mammal, or a mammal in need thereof. In some aspects, the subject is a human, or human in need thereof. In some aspects, the human or human in need thereof is a patient in need of or undergoing medical treatment. In some aspects, the subject can be any age, for example, from about 0 years of age to about 99 years of age, or older.

The term “tooth surface” generally means any surface of a tooth, which can, for example, be an exterior surface of a tooth, including the buccal, lingual, mesial, distal, occlusal, crown, root, or any other common surface designation understood by one of ordinary skill in the art with respect to teeth. Additionally, the term “tooth surface” will be understood as encompassing any internal surface (e.g. root canal wall (s), isthmuses, fins, pulpal chamber wall(s), etc.) of a tooth, which may be accessible through means such as drilling, reaming, or any other method common within the field of dentistry. For brevity, the term “tooth surface” will be understood to encompass any surface, interior or exterior, of a tooth or tooth substitute (e.g. prosthetic).

The term “in vivo” generally means in a living subject.

The term “endodontic therapy” is synonymous with “root canal treatment”, “endodontic treatment”, and “root canal therapy”, and generally refers to a dental procedure for treating a tooth with an infected pulp. It will be appreciated, however, that the tooth need not have infected pulp, i.e. these types of treatments can also be performed on non-infected teeth in certain clinical situations.

In certain aspects of the embodiment, the term “medicament” is synonymous with “irrigant”, “irrigation solution”, “endodontic solution”, and “endodontic irrigant”. In these situations, the medicament is used within the root canal of a tooth as an endodontic irrigant/irrigating solution. In certain aspects of the embodiment, the medicament is created by the mixture of at least two components.

The term “composition,” as used herein, can be used interchangeably with the term “formula.”

The term “chelating agent” generally refers to chemical compounds/molecules that complex with metal ions to form a stable, water-soluble complex. The term “chelating agent” is synonymous with the terms “chelant”, “chelator”, “metal complexing agent”, “complexing agent”, “sequestrant”, and “sequestering agent.”

The abbreviation “EDTA” will be understood to refer to the chemical ethylenediaminetetraacetic acid, which is a chelating agent capable of binding a variety of metal ions, for example, calcium ions. In some aspects of the present disclosure, EDTA is used at a strength of about 17% (w/w).

The term “smear layer” generally refers to the complex accumulation of soluble and insoluble organic and inorganic debris resulting from the mechanical preparation of a tooth surface. Although a smear layer can form on any mechanically prepared tooth surface, the term generally refers to smear layer present within the root canal system following instrumentation. As the smear layer can harbor harmful bacteria, its removal is considered advantageous during endodontic therapy.

The present disclosure describes an aqueous endodontic solution having at least two (i.e. two or more) components, which can be referred to as a medicament. The aqueous endodontic solution can be prepared at a point of use, for example, for irrigating, debriding, and/or disinfecting prepared tooth surfaces, such as root canals. In certain embodiments, the endodontic solution or medicament includes two components. In certain other embodiments, the endodontic solution or medicament consists of two components. The compositions disclosed herein are useful, without limitation, for removing the buildup of undesirable debris formed during the preparation of tooth surfaces during dental procedures, for example, inorganic dentin debris consisting of hydroxyapatite. In certain embodiments, the composition can be used at an appropriate pH to dissolve organic matter and further disinfect the tooth surface. In some embodiments, the composition includes a first component that comprises a partially neutralized phosphonate, such as 2-phosphonobutane-1,2,4-tricarboxylic acid (PBTC), dissolved in an aqueous carrier in one component; and a second component that comprises a hypohalogen oxidizing agent, such as sodium hypochlorite (NaOCl), dissolved in an aqueous carrier. When mixed together, the resultant medicament solution has a pH above 6.2. In some embodiments, where the medicament is to be used for endodontic treatments, the resultant mixed solution has a pH above 8. Without being bound to theory, solutions having a pH above 8 can be used to support tissue dissolution, in addition to disinfection and inorganic debris removal. In short, the compositions and medicaments described herein simultaneously remove organic and inorganic debris from tooth surfaces, such as, for example, a root canal.

The composition of the invention useful for irrigating prepared tooth surfaces is an aqueous composition that generally includes at least one oxidizing agent for the solubilization of organic matter and disinfection, and at least one calcium complexing agent for the removal of inorganic material present within the smear layer. In some embodiments, the oxidizing agent is capable of eradicating bacterium and fungi from the prepared tooth surface.

The oxidizing agent contained in the medicament is preferably a hypohalogen salt, namely sodium hypochlorite (henceforth denoted as “NaOCl”). It will be understood that, in order to minimize degradation and decomposition of the oxidizing agent, the latter may have to be added to the other components of the medicament shortly before application thereof in a dental treatment.

As noted above, in addition to effectively disinfecting the tooth surface and solubilizing organic matter, the composition further removes inorganic materials present in smear layer. Smear layer removal is achieved by the composition, at least in part, by the presence of phosphonates and/or polyphosphates, which may be used for dentin debridement during or after mechanical tooth preparation. The high calcium binding capacity of phosphonates and polyphosphates, in combination with their minimal interaction with oxidizing agents, quickly and thoroughly remove the inorganic components of smear layer. It will be understood that in view of the intended applications, the phosphonates and polyphosphates shall be chosen so as to be safe (well-tolerated, low/acceptable toxicity, etc.) and generally suited for incorporation in an aqueous-based medicament solution.

In some aspects, phosphonates, polyphosphates, carboxylic acids, and sulfonates may be useful for the manufacturing of the disclosed compositions, specifically within the first component of the medicament, to be used for a variety of mechanical treatments of dental hard tissues. Such treatments include, but are not limited to, tooth preparation for a prosthetic application, root canal therapy, and caries excavation. Phosphonates in particular may be used when said medicament is used for removing a dental smear layer produced in a mechanical treatment, for example debridement of dental hard tissue such as prepared root canals.

One such phosphonate is 2-phosphonobutane-1 2 4-tricarboxylic acid (“PBTC”). PBTC is a scale inhibitor that is relatively stable with chlorine, and in particular, hypochlorite. PBTC is a phosphonate that has good calcium complexing properties and does not cause immediate degradation of any oxidizing agent to be used during dental mechanical treatment.

The medicament may also include surfactants to enhance wetting, solubilization (solubility, cleaning and soil removal), debris suspension, emulsification and calcium suspension. The surfactant may be present in the first component of the medicament or in the second component of the medicament, or both components. Examples of suitable wetting agents are anionic and nonionic surfactants such as alkyldiphenyloxide disulfonates, alkyl aryl sulfonates, alkyl sulfates, alcohol ethoxylates, polyoxyethylene glycol octylphenol ethers, polyoxyethylene glycol alkylphenol ethers, polyoxyethylene glycol sorbitan alkyl esters, sorbitan alkyl esters, copolymers of polyethylene glycol and/or propylene glycol, Poloxamers, sodium stearates, sodium lauryl ether sulfates, linear alkylbenzene sulfonates, and amine oxides.

Hydrotropes can also be added to surfactants for formulation stability and to reduce phase separation. Certain hydrotropes that can be used in the medicament include, but are not limited to, sodium xylene sulfonate (SXS), ethylhexyl sulfonate (EHS), and sodium cumene sulfonate (SCS), among others. In one aspect, the hydrotrope is present in the component with the surfactant. For example, when the surfactant is in the first component, the hydrotrope would also be in the first component. In another aspect, the hydrotrope may be present in a component of the medicament without the surfactant.

In one aspect of the medicament, the phosphonate may be present in an amount of 2% to 40% by weight, or 3% to 15% by weight. In one aspect, the first component may be combined with the second component to yield the medicament which has phosphonate content of 2% to 40% by weight, or 3% to 15% by weight.

In one aspect, the final medicament is a solution having a pH greater than 6.2. In some embodiments, the first component contains PBTC, and the second component contains sodium hypochlorite (NaOCl). In some embodiments, the first component of the medicament includes PBTC adjusted with sodium hydroxide (NaOH) so that, when combined with the second component, which includes NaOCl, the resulting medicament is a solution having a pH greater than 7.5.

The working examples set out herein are not to be construed as limiting the scope of this disclosure in any manner. The composition disclosed herein may be useful for irrigating prepared tooth surfaces and may be an aqueous composition that includes an oxidizing agent for the dissolution of tissue and bactericidal disinfection, and a calcium complexing agent for the removal of inorganic parts of smear layer. The oxidizing agent contained in the medicament may be sodium hypochlorite (“NaOCl”). In order to reduce the level of degradation of the oxidizing agent, the latter may have to be added to the other components of the medicament shortly before application thereof in a dental treatment.

The calcium complexing agent may be aqueous 2-phosphonobutane-1,2,4,-tricarboxylic acid (PBTC) adjusted with sodium hydroxide (NaOH) so that when combined with the NaOCl-containing component, the resulting medicament solution has a pH greater than or equal to 8. The medicament may include a combination of additional builders and sequestrants, such as sodium tripolyphosphate (STPP), citric acid, and polyacrylic acids. To assist in cleansing and sequestration, a combination of surfactants (anionics/zwitterionics, and/or nonionics), and hydrotropes may also be added to the formula. The anionics/zwitterionics assist in stabilizing the formula while also contributing to cleansing. They include, but are not limited to, amine oxides, such as Ammonyx; betaines, such as chembetaine; alky aryl sulfonates, such as sodium dodecylbenzene sulfonate; olefin sulfonates, such as sodium lauryl sulfate, and disulfonates, such as Dowfax C6L and C10L. The nonionics assist with anionic surfactant performance and may include ethoxylated alcohols, such as Tomadol and Triton X-100. Hydrotropes may also be added to reduce the chances of phase separation. Examples include, sodium xylene sulfonate, sodium cumene sulfonate, and ethyl hexyl sulfate. Generally cationic surfactants are not desirable within the composition as they are rapidly degraded by hypohalogen anions, such as the hypochlorite anion. In some embodiments, however, one or more cationic surfactants are included in the composition, where the one or more cationic surfactants remain chemically compatible within the medicament composition for at least 15 minutes.

In some aspects, the medicament includes a thixotropic or thickening agent. The thixotropic or thickening agent, when present, may be included in at least one component, or all components. Examples of suitable thixotropic agents are fumed silica and metallic silicates. Examples of suitable thickening agents are polymers, such as polystyrene, polypropylene, polyethylene, polyacrylates, polyacrylamides, polyvinyl alcohol, and copolymers and surfactant combinations, such as an uncharged surfactant (amine oxide, betaine,) combined with an anionic surfactant from the list of suitable anionic surfactants.

Table 1 below provides ranges of ingredients that may be present in some aspects of a medicament according to the present disclosure. Alternative ranges of ingredients that may be present in some embodiments of a medicament according to the present disclosure are provided in Table 1A, below. The chemicals of the table below may be included in at least one components or all components. Additionally, for a preferred two component medicament, Table 2, Table 3 and Table 4 provide example formulas considered by the present disclosure for the first component, second component and mixed medicament, respectively.

In certain embodiments of the invention, a first component has a pH between 10-14. In some embodiments, a first component has a pH between 11-13.5. In some embodiments, a first component has a pH between 12-13.5. Additionally, in some embodiments, the first component has a NaOCl concentration between 4%-12%. In some embodiments, the first component has a NaOCl concentration between 6%-8%.

In some aspects of the invention, the second component has a pH between 6-10. In some embodiments, the second component has a pH between 7-9. In some embodiments, the second component has a pH between 8-8.75.

In certain embodiments of the invention, the medicament resulting from the mixture of the first and second components has a pH between 6-12. In some embodiments, the medicament resulting from the mixture of the first and second components has a pH between 7-10. In some embodiments, the medicament resulting from the mixture of the first and second components has a pH between 8.5-10. In some embodiments, the medicament resulting from the mixture of the first and second components has a pH between 9-10. Illustrated more generally, the medicament resulting from the mixture of the first component and second component shall have an appropriate pH for solubilizing/removing organic and inorganic debris. In additional embodiments of the invention, the medicament resulting from the mixture of the first component and second component has a hypochlorous acid (HOCl) concentration of 0.001% -5%. In some embodiments of the invention, the medicament resulting from the mixture of the first component and second component has a HOCl concentration of 0.005%-0.5%. In some embodiments of the invention, the medicament resulting from the mixture of the first component and second component has a HOCl concentration of 0.01%-0.1%. In some embodiments, when compared to the concentration of the hypochlorite anion, the HOCl concentration is at a ratio of 1/20-1/100,000. In some embodiments, when compared to the concentration of the hypochlorite anion, the HOCl concentration is at a ratio of 1/200-1/20,000. In some embodiments, when compared to the concentration of the hypochlorite anion, the HOCl concentration is at a ratio of 1/1000-1/10,000. In yet other embodiments of the invention, the medicament resulting from the mixture of the first component and second component maintains a NaOCl concentration greater than or equal to 2% for at least 24 hours. In yet other embodiments of the invention, the medicament resulting from the mixture of the first component and second component maintains a NaOCl greater than or equal to 2% for at least 12 hours. In yet other embodiments of the invention, the medicament resulting from the mixture of the first component and second component maintains a NaOCl greater than or equal to 2% for at least five hours. In yet other embodiments of the invention, the medicament resulting from the mixture of the first component and second component maintains a NaOCl greater than or equal to 2% for at least one hour.

In certain embodiments of the invention, at least one component has a pH between 10 and 14. In other embodiments, at least one component has a pH between 11 and 13.5. In other embodiments, at least one component has a pH between 12 and 13.5. Additionally, in some embodiments, the at least one component has a NaOCl concentration between 4% and 12%. In some embodiments, the at least one component has a NaOCl concentration between 6% and 8%.

In some aspects of the invention, a second component, distinct from the first component, has a pH between 6 and 10. In other embodiments, the second component has a pH between 7 and 9. In other embodiments, the second component has a pH between 8 and 8.75.

In certain embodiments of the invention, the medicament resulting from the mixture of the at least two components has a pH between 6 and 12. In some embodiments, the medicament resulting from the mixture of the at least two components has a pH between 7 and 10. In some embodiments, the medicament resulting from the mixture of the at least two components has a pH between 8.5 and 10. In some embodiments, the medicament resulting from the mixture of the at least two components has a pH between 9 and 10. Illustrated more generally, the medicament resulting from the mixture of the first component and second component shall have an appropriate pH for solubilizing/removing organic and inorganic debris. In additional embodiments of the invention, the medicament resulting from the mixture of the at least two components has a hypochlorous acid (HOCl) concentration of 0.001%-5%. In some embodiments, the medicament has a HOCl concentration of 0.005%-0.5%. In some embodiments, the medicament has a HOCl concentration of 0.01%-0.1%. In yet other embodiments of the invention, the medicament resulting from the mixture of the first component and second component preferably maintains a NaOCl concentration greater than or equal to 2% for at least 24 hours. In some embodiments, the medicament preferably maintains a NaOCl concentration greater than or equal to 2% for at least 12 hours. In some embodiments, the medicament preferably maintains a NaOCl concentration greater than or equal to 2% for at least five hours.

In some embodiments, the medicament is prepared via a 1:1 (50%/50%) mix of the first component and second component. However, it is understood that the component chemistries may be modified such that alternative mix ratios, and total number of components, may be implemented to yield the same mixed medicament. These alternative mix ratios could comprise any mix ratio between at least two components but ideally the mixing would entail at least 5% of one component to facilitate ease of measuring and mixing.

TABLE 1

| Ingredients | w/w % |
|---|---|
| Sodium hypochlorite | 0.1-14% |
| Deionized Water | 8.95-95% |
| 2-phosphonobutane-1,2,4- | 2-40% |
| Tricarboxylic acid | |
| Acrylic acid homopolymer | 0.5-10% |
| Sodium Hydroxide | 0.1-10% |
| Citric Acid | 0-5% |
| Sodium Tripolyphosphate | 0-5% |
| Tetra Potassium Pyrophosphate | 0-5% |
| Phytic Acid | 0-30% |
| Sodium Dodecylbenzenesulfonate | 0-5% |
| Alkyldiphenyloxide Disulfonate | 0-7% |
| Secondary Alcohol Ethoxylate | 0-2.5% |
| Amine oxide | 0-5% |
| Polyethylene glycol tert- | 0-5% |
| octylphenyl ether | |
| Sodium Xylene Sulfonate | 0-5% |
| Sodium Lauryl Sulphate | 0-2.5% |
| Ethyl Hexyl Sulfate | 0-2.5% |
| Sodium Cumene Sulfonate | 0-2.5% |
| Sodium Chloride | 0-5% |

TABLE 1A

| Ingredients | w/w % |
|---|---|
| Sodium hypochlorite | 0.1-14% |
| Deionized Water | 8.95-95% |
| 2-phophonobutane-1,2,4- | 2-40% |
| Tricarboxylic acid | |
| Acrylic acid homopolymer | 0.5-2% |
| Sodium Hydroxide | 0.1-7% |
| Citric Acid | 0-2% |
| Sodium Tripolyphosphate | 0-2% |
| Tetra Potassium Pyrophosphate | 0-2% |
| Phytic Acid | 0-30% |
| Sodium Dodecylbenzenesulfonate | 0-3% |
| Alkyldiphenyloxide Disulfonate | 0-7% |
| Secondary Alcohol Ethoxylate | 0-2% |
| Amine oxide | 0-1% |
| Polyethylene glycol tert- | 0-2% |
| octylphenyl ether | |
| Sodium Xylene Sulfonate | 0-4% |
| Sodium Lauryl Sulphate | 0-1% |
| Ethyl Hexyl Sulfate | 0-1% |
| Sodium Cumene Sulfonate | 0-1% |
| Sodium Chloride | 0-3% |

Table 2 provides example formulations for a first component containing at least one oxidizing agent.

TABLE 2

| | Formula | Formula | Formula | Formula | Formula |
|---|---|---|---|---|---|
| | 1.1 | 1.2 | 1.3 | 1.4 | 1.5 |
| Ingredient | w/w % | w/w % | w/w % | w/w % | w/w % |
| Deionized Water | 89.90% | 79.30% | 80.17% | 87.05% | 90.63% |
| Sodium hypochlorite | 8.10% | 8.10% | 8.10% | 8.10% | 1.10% |
| Dodecylbenzenesulfonate (79.3%) | 0.00% | 0.68% | 0.00% | 0.00% | 0.00% |
| DowFax C6L | 0.00% | 2.28% | 2.28% | 0.00% | 2.28% |
| DowFax C10L | 0.00% | 0.84% | 0.84% | 0.00% | 0.84% |
| Acusol 445 | 0.00% | 3.30% | 3.30% | 0.00% | 3.30% |
| Sodium hydroxide (50% sol) | 2.00% | 1.80% | 2.16% | 2.00% | 1.00% |
| Tomadol 91-6 | 0.00% | 0.51% | 0.51% | 0.51% | 0.51% |
| Ammonyx LO | 0.00% | 0.34% | 0.34% | 0.34% | 0.34% |
| Sodium Xylene Sulfonate | 0.00% | 2.30% | 2.30% | 0.00% | 0.00% |
| Sodium Lauryl Sulphate | 0.00% | 0.25% | 0.00% | 0.00% | 0.00% |
| Ethyl Hexyl Sulfate | 0.00% | 0.30% | 0.00% | 0.00% | 0.00% |
| Sodium Chloride | 0.00% | 0.00% | 0.00% | 1.00% | 0.00% |
| Sodium Chlorate | 0.00% | 0.00% | 0.00% | 1.00% | 0.00% |
| Total | 100.00% | 100.00% | 100.00% | 100.00% | 100.00% |
| Target pH (+/−0.5) | 13.1 | 12.9 | 12.7 | 12.8 | 11.2 |

Table 3 provides example formulations for the second component containing at least one metal complexing agent, and more specifically, at least one calcium complexing agent.

TABLE 3

| | Formula | Formula | Formula | Formula | Formula | Formula |
|---|---|---|---|---|---|---|
| | 2.1 | 2.2 | 2.3 | 2.4 | 2.5 | 2.6 |
| Ingredient | w/w % | w/w % | w/w % | w/w % | w/w % | w/w % |
| Deionized Water | 41.88% | 41.38% | 39.88% | 38.88% | 47.36% | 50.77% |
| Dequest 7000 | 25.79% | 25.79% | 25.79% | 25.79% | 25.79% | 25.79% |
| Acusol 445 | 3.30% | 3.30% | 3.30% | 3.30% | 3.30% | 0.00% |
| Sodium Hydroxide (50% Sol.) | 18.70% | 18.70% | 18.70% | 18.70% | 10.70% | 17.50% |
| Citric Acid | 0.59% | 0.59% | 0.59% | 0.59% | 0.59% | 0.59% |
| Sodium Tripolyphosphate | 1.52% | 1.52% | 1.52% | 1.52% | 1.52% | 1.52% |
| Tetra Potassium Pyrophosphate | 0.00% | 0.00% | 0.00% | 0.00% | 1.52% | 0.00% |
| Dodecylbenzenesulfonate (79.3%) | 0.68% | 0.68% | 0.68% | 0.68% | 0.68% | 0.68% |
| DowFax C10L | 0.84% | 0.84% | 0.84% | 0.84% | 0.84% | 0.00% |
| DowFax C6L | 2.28% | 2.28% | 2.28% | 2.28% | 2.28% | 0.00% |
| Tomadol 91-6 | 0.51% | 0.51% | 0.51% | 0.51% | 0.51% | 0.00% |
| Ammonyx LO | 0.34% | 0.34% | 0.34% | 0.34% | 0.34% | 0.00% |
| Triton X-100 | 0.42% | 0.42% | 0.42% | 0.42% | 0.42% | 0.00% |
| Sodium Xylene Sulfonate | 2.30% | 2.30% | 2.30% | 2.30% | 2.30% | 2.30% |
| Sodium Lauryl Sulphate | 0.25% | 0.25% | 0.25% | 0.25% | 0.25% | 0.25% |
| Ethyl Hexyl Sulfate | 0.30% | 0.30% | 0.30% | 0.30% | 0.30% | 0.30% |
| Sodium Cumene Sulfonate | 0.30% | 0.30% | 0.30% | 0.30% | 0.30% | 0.30% |
| Sodium Chloride | 0.00% | 0.50% | 2.00% | 3.00% | 3.00% | 0.00% |
| Total | 100.00% | 100.00% | 100.00% | 100.00% | 100.00% | 100.00% |
| Target pH (+/−0.5) | 8 | 8 | 8 | 8 | 4.2 | 7.5 |

Table 4 provides example formulations of the medicament created by mixing at least one component.

TABLE 4

| | Formula | Formula | Formula | Formula | Formula |
|---|---|---|---|---|---|
| | A | B | C | D | E |
| Ingredient | w/w % | w/w % | w/w % | w/w % | w/w % |
| Deionized Water | 65.87% | 64.87% | 63.87% | 62.87% | 70.61% |
| Sodium hypochlorite | 4.05% | 4.05% | 4.05% | 4.05% | 0.55% |
| Dequest 7000 | 12.90% | 12.90% | 12.90% | 12.90% | 12.90% |
| Acusol 445 | 1.65% | 1.65% | 1.65% | 1.65% | 1.65% |
| Sodium Hydroxide (50% Sol.) | 10.35% | 10.35% | 10.35% | 10.35% | 7.35% |
| Citric Acid | 0.30% | 0.30% | 0.30% | 0.30% | 0.30% |
| Sodium Tripolyphosphate | 0.76% | 0.76% | 0.76% | 0.76% | 0.76% |
| Tetra Potassium Pyrophosphate | 0.00% | 0.00% | 0.00% | 0.00% | 0.76% |
| Dodecylbenzenesulfonate (79.3%) | 0.34% | 0.34% | 0.34% | 0.34% | 0.34% |
| DowFax C10L | 0.42% | 0.42% | 0.42% | 0.42% | 0.42% |
| DowFax C6L | 1.14% | 1.14% | 1.14% | 1.14% | 1.14% |
| Tomadol 91-6 | 0.26% | 0.26% | 0.26% | 0.26% | 0.26% |
| Ammonyx LO | 0.17% | 0.17% | 0.17% | 0.17% | 0.17% |
| Triton X-100 | 0.21% | 0.21% | 0.21% | 0.21% | 0.21% |
| Sodium Xylene Sulfonate | 1.15% | 1.15% | 1.15% | 1.15% | 1.15% |
| Sodium Lauryl Sulphate | 0.13% | 0.13% | 0.13% | 0.13% | 0.13% |
| Ethyl Hexyl Sulfate | 0.15% | 0.15% | 0.15% | 0.15% | 0.15% |
| Sodium Cumene Sulfonate | 0.15% | 0.15% | 0.15% | 0.15% | 0.15% |
| Sodium Chloride | 0.00% | 0.50% | 2.00% | 3.00% | 0.00% |
| Sodium Chlorate | 0.00% | 0.50% | 0.00% | 0.00% | 1.00% |
| Total | 100.00% | 100.00% | 100.00% | 100.00% | 100.00% |
| Target pH (+/−0.5) | 9.5 | 9.5 | 9.5 | 9.5 | 7.5 |

### Making the Medicament

In general, to make the first and second components of a medicament according to the aspects of the present disclosure in a first step, the acid ingredients of the component may first be dissolved or dispersed in water to make an aqueous solution or dispersion. In a second step, any builders to be used, such as those listed above, may be dissolved or dispersed in water. In a third step, the acids and builders may be neutralized to a more neutral pH, for example, using a base (for example, such as sodium hydroxide, potassium hydroxide, or other alkali salts). In a fourth step, the surfactants and/or hydrotropes, if any, may be added to the neutralized solution or dispersion. In the fifth step, final pH adjustments are made to achieve the target pH using acids (such as hydrochloric acid, sulfuric acid, perchloric acid, chloric acid, or other acids) and bases (such as sodium hydroxide, potassium hydroxide, or other alkali salts). Throughout all steps moderate agitation may be employed, along with maintaining the solution between 50° F. and 180° F. In some embodiments, the solution is maintained at a temperature between 50° F. and 140° F. In some embodiments, the solution is maintained at a temperature between 50° F. and 120° F. A person of skill in the art will appreciate that some or all of these steps may be practiced in a different order to yield the formulation.

### Packaging of Product

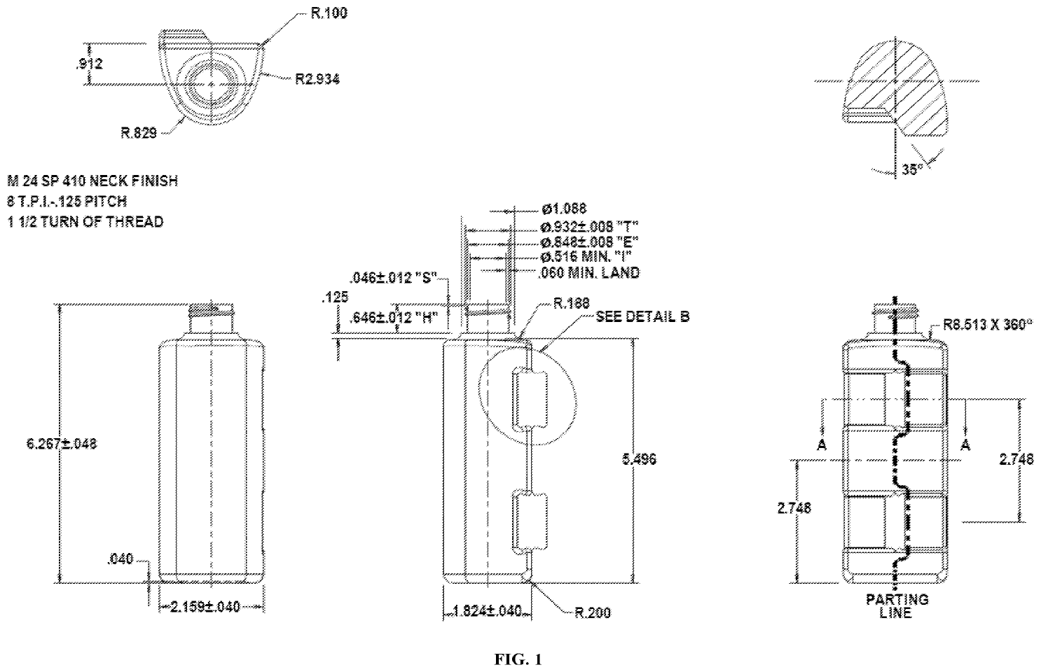

In some aspects of the present disclosure, the at least two components may be provided in separate containers, packaged so that they can be mixed to yield the final medicament at the time of the procedure. FIG. 1 depicts a bottle capable of interlocking with itself to form two separate containers that when slide together creates a single bottle with two individual compartments. In one aspect, the first component can be provided in a first container or first bottle, and the second component can be provided in a second container or second bottle. Optionally, one or both bottles may be provided with sufficient empty volume so that the first component may be poured directly into the second bottle, or vice versa. In another aspect, the two bottles may be combined in a third container.

In certain embodiments of the invention, two interlocking bottles are used to separately store each component and a cap is attached to the bottles for means of dispensing the fluid to the user. This cap comprises an external housing piece and internal fluid connection means. The fluid connection means is comprised of tubing and valves to separately, yet simultaneously, extract solution from each individual interlocking bottle to an internal mixing chamber which then leads to a luer activated valve for easily filling syringes. Through this process, the medicament components self-mix within the mixing chamber to then be extracted by the user via a syringe. To mitigate the potential for cross-contamination of one component into the other component's bottle, one way check valves may be included within the fluid connection means. Additionally, due to the limited stability of the medicament after mixing, it is advantageous to minimize the amount of fluid space within the cap where the components are mixed (i.e. the fluid space after the check valves to the luer activated valve). In some embodiments, this fluid space is less than 1.5 milliliters. In some embodiments, this fluid space is less than one milliliter. In some embodiments, this fluid space is less than 500 microliters. In some embodiments, the mixing chamber is a wye or tee fitting or similar design.

In other embodiments of the invention, a container, vessel, or other storage package is provided for each of the individual components which are then combined to prepare the irrigant/medicament. In some embodiments, the components are aqueous-based liquids for quick mixing and homogenization. In other embodiments, at least one component is a powder and at least one other component is an aqueous-based liquid.

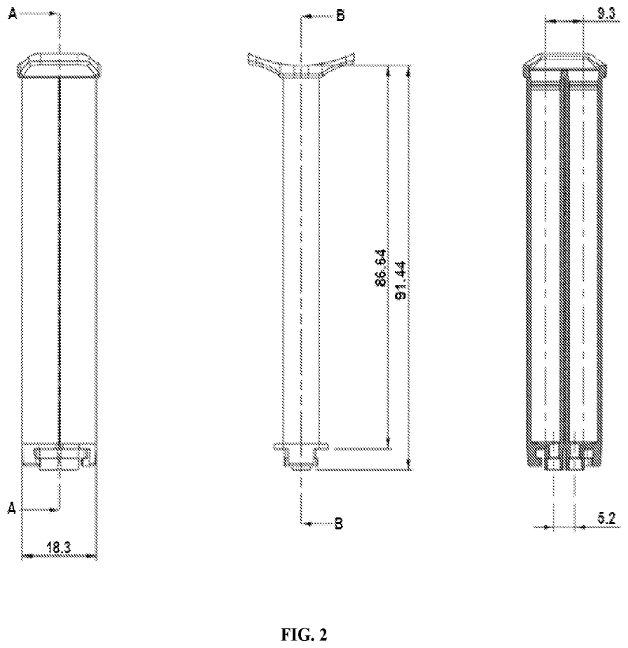

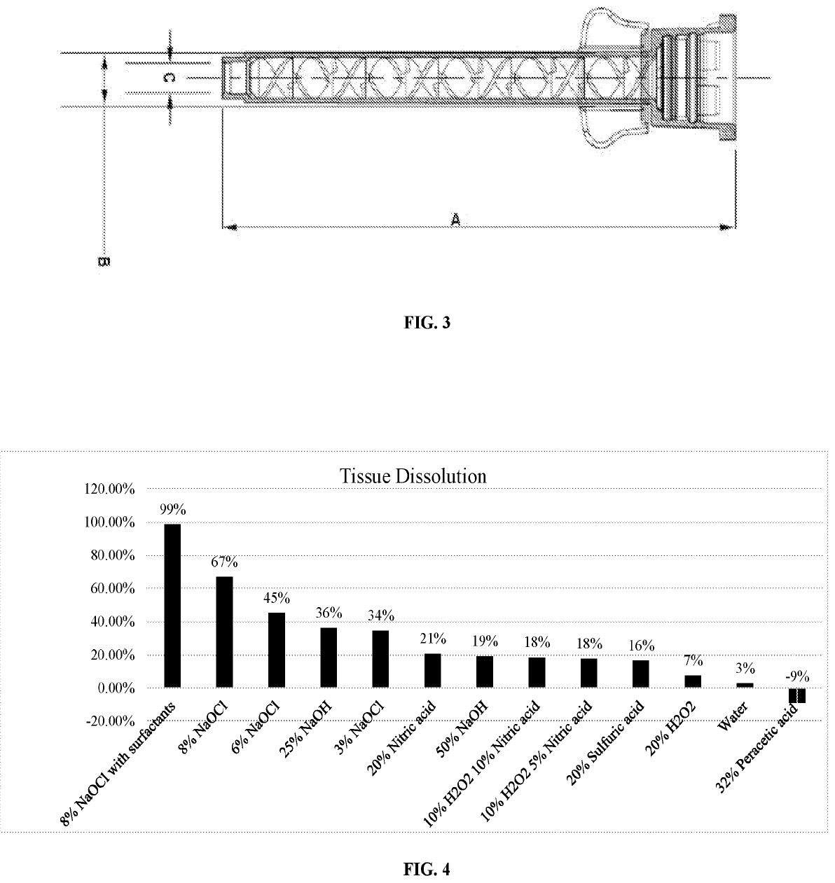

In another aspect, a dual-barrel syringe may be used to store the first component and the second component prior to use. For example, FIG. 2 shows such a syringe that includes a first barrel and a second barrel separate from the first barrel. The first component may be stored in the first barrel and the second component may be stored in the second barrel. When a user operates the plunger or plungers of the dual barrel syringe, the first component and the second component may mix in a volume which is either contained within the syringe, or outside of the syringe, forming the final medicament. In such a situation, the dispensing system (i.e. dual barrel syringe) may additionally comprise the delivery system (i.e. application to the treatment site). This is in contrast to the interlocking dual chamber bottles discussed previously which only comprise the dispensing system, as other delivery systems (e.g. separate syringes) would be needed for applying the medicament to the treatment site. FIG. 3 shows a mixing tip that can be connected to the dual barrel syringe capable of controlling dispensing and ensure adequate mixing of the first component and second component.

The packaging of the components must be compatible for long-term storage (months to years). Satisfactory plastic resins for the packaging material may include, but are not limited to, polypropylene, polyethylene, styrene acrylonitrile, methyl methacrylate-acrylonitrile-butadiene-styrene, poly-cyclohexylenedimethylene terephthalate glycol, among others.

In some aspects, the medicament is provided in a kit, wherein the first component is stored separately (i.e. not in fluid contact with) from the second component. In some aspects, the kit includes a dual-barrel syringe. In some aspects, the kit comprises any of the following components: mixing vessels, mixing tips, empty syringes, application tips or brushes, irrigation tips, an instructions for use, an apex locator, an endodontic sealer, burrs, dental handpieces, gutta percha points, endodontic files, paper points, among other common dental and endodontic devices and products.

FIG. 1 is a depiction of a dual chamber bottle that is capable of inter-locking with the opposing half suitable for storing and dispensing the disclosed medicament.

FIG. 2 is a depiction of a dual cartridge syringe that is capable of engaging with a mixing tip suitable for storing and dispensing the disclosed medicament.

FIG. 3 illustrates a mixing tip capable of engaging with a dual cartridge syringe which allows for adequate mixing of the individual components prior to administration at the treatment site.

### Method of Using the Medicament

The present disclosure also teaches methods of using the medicament clinically for debriding and cleansing a tooth surface, such as a root canal in vivo. This method entails mixing at least two components to yield a single medicament capable of simultaneously solubilizing organic and inorganic debris when administered in vivo in the root canal via a syringe and irrigating needle, wherein at least one component contains sodium hypochlorite, wherein at least one separate component contains a calcium chelating agent, wherein the single medicament provides greater than or equal to 2% sodium hypochlorite for at least one hour, and wherein the single medicament is the only irrigant needed to perform an endodontic procedure. The syringes and irrigating needles employed to apply the medicament may be of any type common within the dental and medical fields and may include the use of at least one 1-30 mL syringe, and at least one irrigating needle of varying sizes between 15-32 gauge. In some instances, the irrigating needles may incorporate brushes or other features to aid application.

### Testing

Since the smear layer produced upon mechanical debridement of infected dental hard tissues includes both organic and inorganic dentin components, an investigation was conducted to determine the organic tissue dissolution capacity of various antiseptics and chelating solutions that are commonly used in dentistry and medical applications. For this purpose, soft tissue samples were incubated in aqueous solutions containing the agents to be studied and the mass loss of the tissue samples was measured. The results are collected in FIG. 4, where the average mass loss is given as a percentage fraction of the initial weight. FIG. 4 illustrates the mass loss (Mean %) of tissue samples incubated in 30 mL of aqueous solutions of various agents after 30 minutes.

From the results shown in FIG. 4, sodium hypochlorite (NaOCl) solutions produced the largest mass loss, which may be desirable for organic tissue dissolution. It can be seen that high concentrations of NaOCl along with the addition of surfactants (anionic and nonionic) tended to increase the tissue dissolution rate.

As NaOCl interacts with organic tissue it breaks down the tissue via several reactions, such as: saponification, amino acid neutralization, and chloramination (see Schemes 1-3), among other reactions. In the saponification reaction, NaOCl acts as an organic and fat solvent/dissolution agent by breaking down fatty acids into fatty acid salts (soap) and glycerol, which then reduce the surface tension of the solution. During the amino acid reaction, NaOCl dissolves amino acids forming water and salt. As the water is formed with the hydroxide ion, the pH is also reduced. In the chloramination reaction hypochlorous acid and hypochlorite ions interact with organic tissue which leads to amino acid degradation and hydrolysis. This reaction forms chloramines that interfere with cell metabolism.

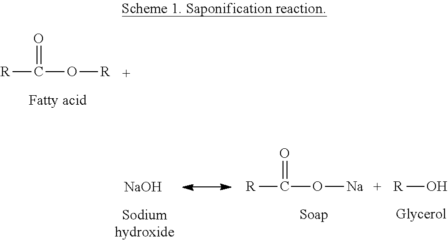

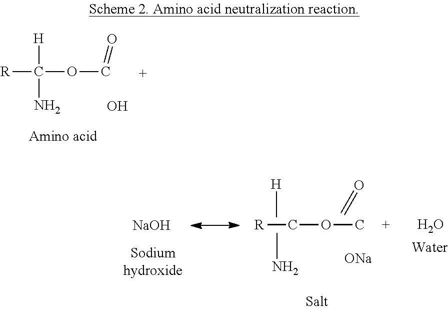

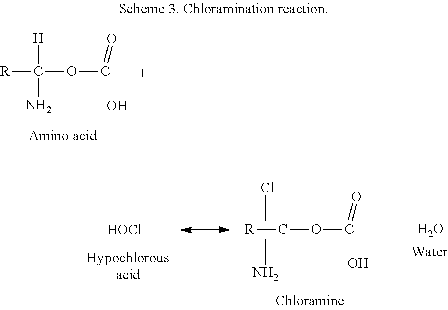

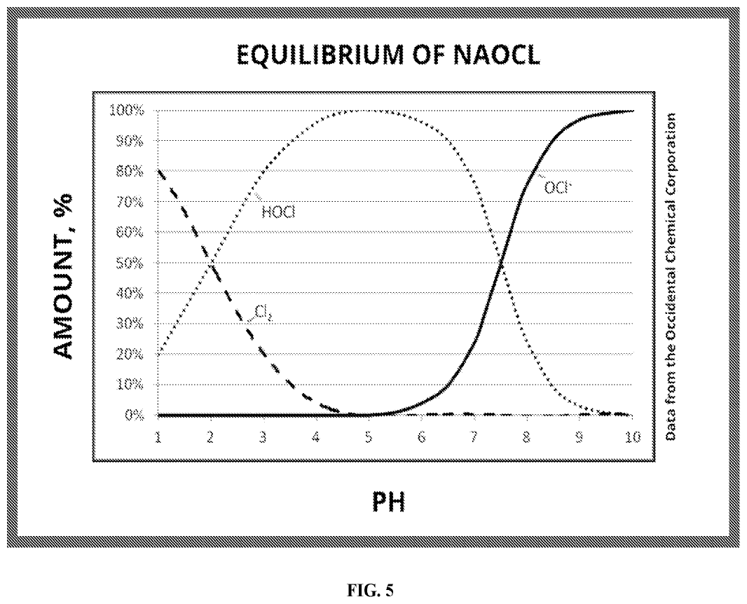

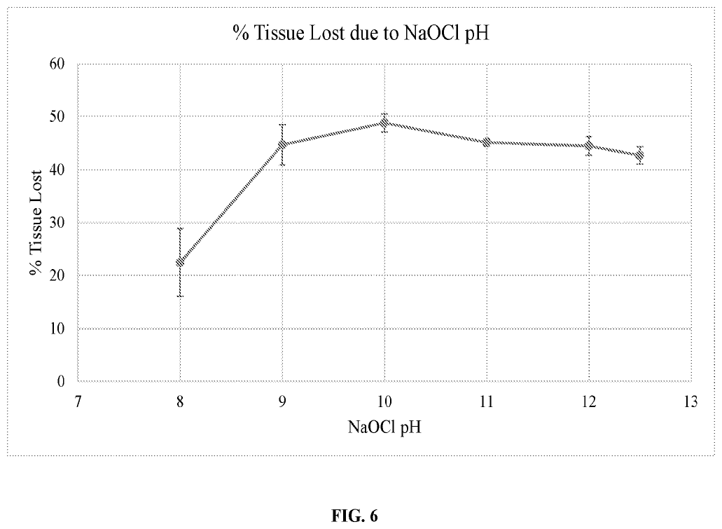

The organic tissue dissolution capacity of NaOCl may be a function of free available chlorine in solution and a function of alkalinity. As pH increases to above 6.2 the proportion of the hypochlorite anion (OCl⁻) to hypochlorous acid (HOCl) dramatically increases. Below this pH, the concentration of hypochlorite anion decreases and the concentration of HOCl increases (see FIG. 5, which illustrates the chlorine speciation profile as a function of pH). As the concentration of HOCl increases, the antimicrobial properties increase as HOCl is 80-100 times more effective than NaOCl as a sanitizer. However, as the concentration of the hypochlorite ions decreases, the tissue dissolution capacity also decreases (see FIG. 6, which illustrates mass loss of tissue samples incubated in 30 mL of sodium hypochlorite at different pH values after 30 minutes.). In certain aspects of the invention, the medicament comprises HOCl and OCl— and has a pH between 8 and 10.

Since NaOCl is an oxidizing agent capable of dissolving organic tissue, and since it was most capable of dissolving organic tissue at a pH above 8, a second investigation was aimed at determining the effect that various chelating agents used in household, institutional, and personal care products have on the amount of free available chlorine in aqueous NaOCl solutions. Table 5 shows the amount of free available chlorine (expressed as percentage fraction of the theoretical maximum) and the pH-value as a function of time in mixtures of aqueous chelating agent solutions and a 3% sodium hypochlorite solution. The agents that were tested are deionized water (as a control), 1-hydroxyethylidene-1,1-diphosphonic acid (HEDP), ethylenediaminetetraacetic acid (EDTA), phytic acid (C₆H₆[OPO(OH)₂]₆) (PA), sodium hexametaphosphate (SH), N-(Phosphonomethyl)iminodiacetic acid (PMIDA), Poly(4-styrenesulfonic acid maleic acid) sodium salt (PSS), and 2-phosphonobutane-1,2,4,-tricarboxylic Acid (PBTC). Table 5 shows the amount of free available chlorine (expressed as percentage of theoretical maximum rounded to the nearest 10%) and pH of different chelating agent solutions mixed in a 1:1 ratio with 3% NaOCl aqueous solution over time.

TABLE 5

| | | | Free Available Chlorine Remaining | | | |
|---|---|---|---|---|---|---|
| | | | Post Mix (hours) | | | |
| Agent | Wt/vol | pH | 0 | 1 | 4 | 24 |
| DI Water | — | 9.1 | 100% | 100% | 90% | 80% |
| HEDP | 15% | 9.2 | 100% | 20% | 10% | 0% |
| HEDP | 15% | 11.1 | 100% | 80% | 40% | 20% |
| EDTA | 17% | 8.5 | 100% | 0% | 0% | 0% |
| PA | 30% | 9.5 | 100% | 90% | 90% | 70% |
| SH | 20% | 10.1 | 100% | 100% | 90% | 20% |
| PMIDA | 15% | 11.5 | 100% | 0% | 0% | 0% |
| PSS | 15% | 11.5 | 100% | 90% | 90% | 90% |
| PBTC | 20% | 9.1 | 100% | 80% | 60% | 40% |

PBTC, phytic acid (PA), sodium hexametaphosphate (SH), PSS, and HEDP above a pH of 11 were able to maintain most of their initial available chlorine in these solutions after 1 hour. Those that were unable to maintain their available chlorine and chlorite had dramatically less organic tissue dissolution capacity. In certain embodiments of the invention, the incorporated chelating agent demonstrates limited stability with NaOCl and results in at least 60% of the initial NaOCl concentration after one hour, and at least 40% of the initial NaOCl concentration after four hours.

Much of the subsequent testing was performed using Formula A (as detailed in Table 4), for illustrative and example purposes, and comparing its performance to typical endodontic irrigation protocols including the sequential use of 3-6% NaOCl and 17% EDTA. Although the subsequent experimental results were prepared using Formula A, one of ordinary skill will appreciate that similar results will be obtained using any of the compositions or formulations disclosed herein.

In a preliminary investigation of smear layer removal, PSS, SH, and PA were found to be relatively weak calcium chelators. As such they were not included in the third investigation, which evaluated the ability of Formula A (from Table 4), HEDP (Dual Rinse®, Medcem, Vienna, Austria), and 17% EDTA to remove smear layer from instrumented root canals. Median smear layer scores in irrigated teeth are shown in Table 6. A score of 1 indicates very little smear layer remaining, whereas a score of 5 indicates heavy smear layer present.

TABLE 6

| | | HEDP + 4.8% | 5.25% | 17% EDTA + 4.8% |
|---|---|---|---|---|
| | Formula A | NaOCl | NaOCl | NaOCl |
| Apical | 1.00 | 1.10 | 3.5 | 1.50 |
| Middle | 1.00 | 1.00 | 3.6 | 1.00 |
| Coronal | 1.00 | 1.00 | 3.2 | 1.00 |

Mean score registered for each group in the different root canal thirds in the evaluation of residual smear layer. A score of 1 indicates very little smear layer remaining, whereas a score of 5 indicates heavy smear layer present.

Formula A at the tested concentration showed statistically significantly better smear layer removal compared to sodium hypochlorite and sodium hypochlorite used with EDTA. The results were not significantly better than HEDP, but the results were comparable. However, in a fourth investigation it was found that Formula A had a marked antimicrobial impact compared to HEDP (Dual Rinse®) with NaOCl, or EDTA with NaOCl (Table 7). Table 7 provides information about the growth of C. Albicans, measured as colony forming units per mL (CFU/mL), after 3-min exposure to the various treatment irrigants/medicaments. Therefore, the disclosed invention can simultaneously remove smear layer from the root canal space, thereby leaving a clean canal, while also providing significant antimicrobial activity, whereas the other irrigants investigated were not able to provide excellent results for both tests. As is evident by these results, the disclosed invention provides marked improvements in endodontic irrigant efficacy when compared to competitive products (e.g. Dual Rinse HEDP) and typical endodontic irrigation regiments using NaOCl and EDTA.

TABLE 7

| Treatment | Average CFU/mL |
|---|---|
| Formula A | 2 |
| 5.25% NaOCl + | 42.5 |
| Dual Rinse HEDP | |
| 5.25% NaOCl + | 419.5 |
| 17% EDTA | |
| Untreated | 250000 |

### Experiment was Performed with n=4 Per Group

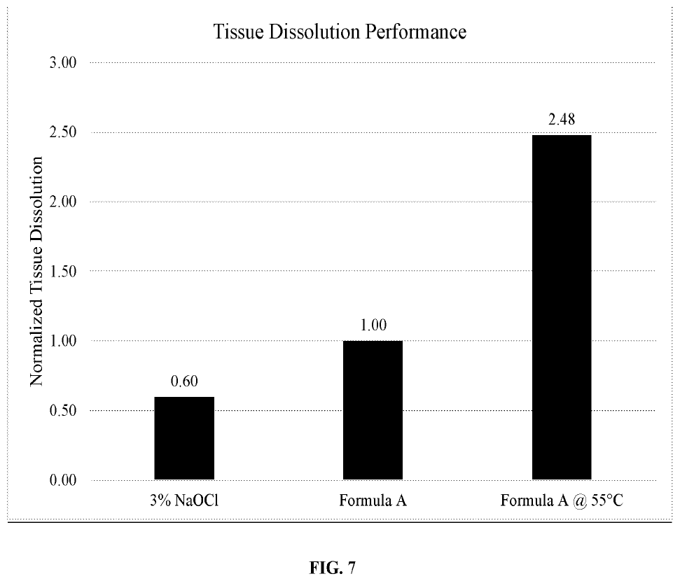

A tissue dissolution experiment was subsequently executed comparing 3% NaOCl to Formula A at room temperature (20° C.) and heated to 55° C. Tissue specimens (0.5 g) were incubated in 10 mL of irrigant for 30 min and the mass loss was calculated using initial and final mass measurements using a calibrated analytical scale. Compared to standard 3% NaOCl at room temperature, Formula A at room temperature dissolved 1.67× more tissue (FIG. 7). Heating of Formula A further increased the tissue dissolution rate and dissolved 2.5× and 4.1× more tissue than Formula A at room temperature and 3% NaOCl, respectively. As such, in preferred embodiments of the invention, the disclosed medicament dissolves 1.5×5× more tissue than standard NaOCl of similar concentration. As such, in more preferred embodiments of the invention, the disclosed medicament dissolves 2.5×4× more tissue than standard NaOCl of similar concentration.

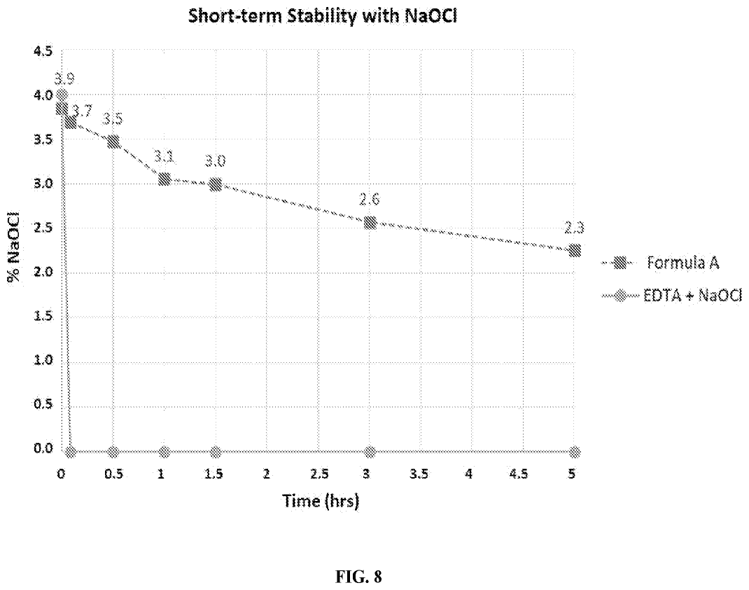

The NaOCl stability of Formula A was evaluated during another experiment and compared to mixing one part 17% EDTA (17% EDTA, Vista Apex, Racine, WI) with one part 8% NaOCl (Sigma Aldrich, Milwaukee, WI). At defined timepoints, samples of the mixtures were obtained and the NaOCl concentration was analytically measured via iodometric titration. Results are shown in FIG. 8. Overall, Formula A was shown to have excellent short-term stability with NaOCl as measured NaOCl levels were greater than or equal to 2% at all timepoints up to five hours. Conversely, EDTA+8% NaOCl was very unstable and resulted in 0% NaOCl in under five minutes of being mixed. Therefore, when the components of the disclosed medicament are mixed, a NaOCl concentration greater than or equal to 2% NaOCl is preferably maintained for at least 24 hours, more preferably maintained for at least 12 hours, and even more preferably maintained for at least five hours.

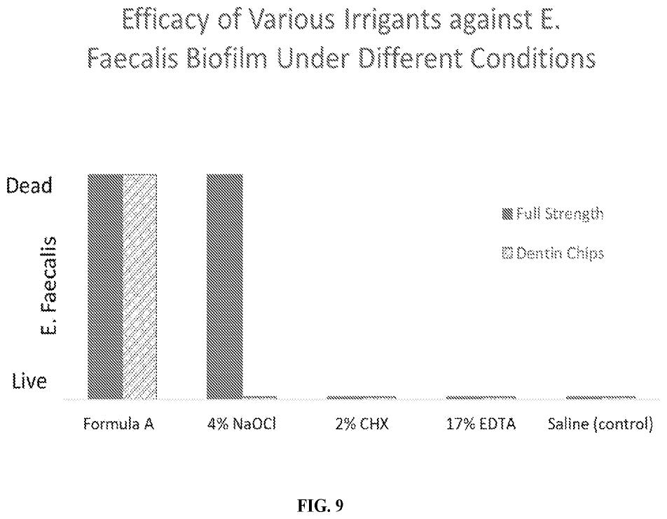

In yet another experiment, the ability of various endodontic irrigants/medicaments to disrupt a biofilm consisting of the bacteria E. faecalis in the presence of dentin chips was investigated. E. faecalis biofilms were grown for two days within 96 well plates following industry standard practices for cell culture. Various endodontic irrigants—Formula A, 4% NaOCl (Sigma Aldrich, Milwaukee, WI), 2% CHX (Vista Apex, Racine, WI), 17% EDTA (Vista Apex, Racine, WI) and normal 0.9% saline (Sigma Aldrich, Milwaukee, WI)—were then applied to the biofilms and the amount of live vs dead bacteria was quantified using laboratory standard live/dead staining and imaging with confocal microscopy. As an additional test group, the effect of dentin powder was investigated by incubating 20 mg of dentin powder with 1 mL of endodontic irrigant for 10 minutes prior to exposure to the biofilm. Results are shown in FIG. 9. As the results show, Formula A was the only irrigant/medicament that was effective at killing the biofilm in the presence or absence of dentin. Conversely, the presence of dentin inhibited and/or neutralized the efficacy of all other irrigants studied. This experiment shows the significant utility of the invention as no other endodontic irrigant was able to maintain its clinical efficacy in the presence of dentin. In preferred embodiments of the invention, the medicament retains its antimicrobial activity in the presence of dentin.

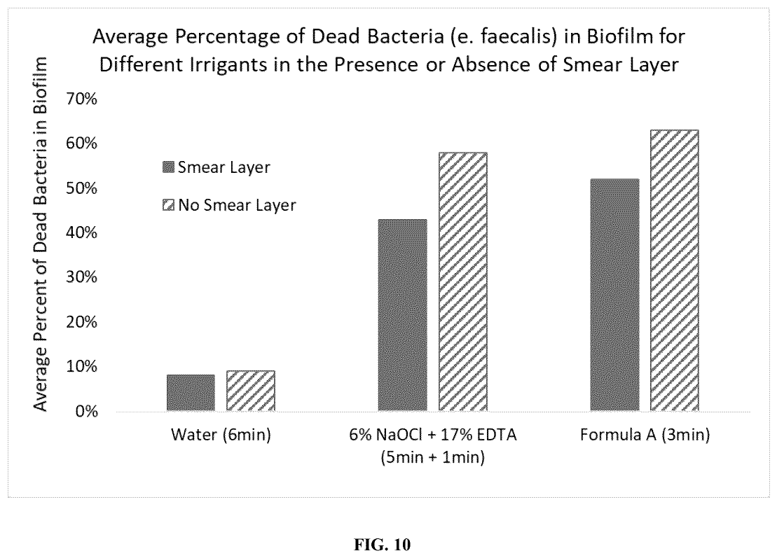

An additional antimicrobial study was executed to investigate the invented medicament's ability to remain clinically effective in the presence of smear layer and a mature biofilm. E. faecalis biofilms were established on dentin blocks following published literature (J Endod. 2011 October; 37(10):1380-5.) and allowed to mature for three weeks creating test specimens. Following incubation, a smear layer was created on a subset of the specimen samples using a medium-grit cylinder flat-end bur (Patterson Dental, Halifax, Canada) at 1500 rpm for 4 seconds each. The other specimen samples remained unaltered to mimic a “no smear layer” condition. 100 uL of each experimental medicament/irrigant was applied to the smear layer specimens and non-smear layer specimens for various durations. The medicament/irrigant was aspirated away, and the specimens were rinsed with sterile saline water. The specimens were then sectioned perpendicular to the surface, and the number of live/dead bacteria within the dentinal tubules was quantified using laboratory standard live/dead staining and imaging with confocal microscopy. Results are shown in FIG. 10, which illustrates that the invented medicament (Formula A) was more effective than the standard endodontic irrigation regiment (6% NaOCl for 5min plus 17% EDTA for 1 min) in half the time. In other words, the medicament was able to provide more efficacious results compared to the standard irrigation regiment while also saving time. Results were consistently better for Formula A in the presence or absence of smear layer.

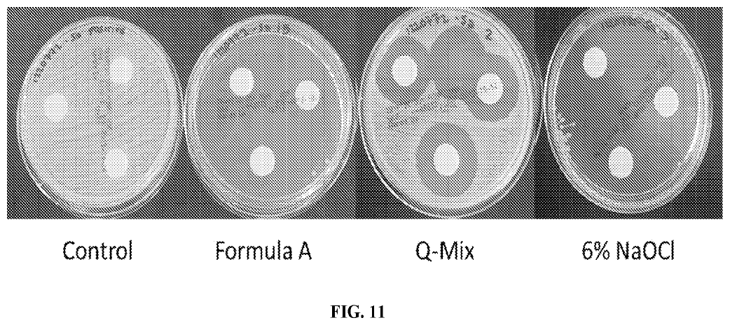

Another antimicrobial study was performed by Nelson Laboratories (Salt Lake City, UT). The test is called “Antimicrobial Susceptibility Test: Zone of Inhibition” and followed Nelson Laboratories' controlled procedure STP0124 (Rev 2, Update 1) which is based on various laboratory standard methods (Murray et al. Manual of Clinical Microbiology. 2007. 9^{th} Ed. Chapter on Antibacterial Susceptibility Tests: Dilution and Disk Diffusion Methods, and M02-A11. Vol. 32 No. 1. 2012. Performance Standards for Antimicrobial Disk Susceptibility Tests; Approved Standard—Eleventh Edition. Clinical and Laboratory Standards Institute (CLIS), Wayne, PA. CRD411.). Briefly, inoculated agar plates (using a bacterial cell density equivalent to 0.5 McFarland standard) were subjected to various endodontic irrigants (sterile water as a control, Q-Mix (Dentsply, PA) and 6% NaOCl (Vista Apex, Racine, WI) and Formula A) via a sterile disk saturated with the irrigant as detailed in the aforementioned procedures/standards. A “zone of inhibition” (i.e. area of disrupted bacterial growth) was then observed around the disk saturated with the irrigant. Results are shown in FIG. 11 where it is clearly visible that Formula A provided the greatest zone of inhibition compared to the other experimental irrigants.

The present description is for illustrative purposes only and should not be construed to narrow the breadth of the present disclosure in any way. Thus, those skilled in the art will appreciate that various modification might be made to the presently disclosed embodiments without departing from the full and fair scope and spirit of the present disclosure. Other aspects, features, and advantages will be apparent upon an examination of the attached drawings and appended claims.

### Statements

The following statements are illustrative and within the scope of the embodiments of the invention described herein.

Statement 1: A method of irrigating a root canal, comprising:
mixing of at least two components to yield a single medicament capable of simultaneously solubilizing organic and inorganic debris; and,
administering the medicament in vivo in the root canal via a syringe and irrigating needle;
wherein at least one component contains sodium hypochlorite;
wherein at least one separate component contains at least one calcium chelating agent.

Statement 2: The method according to statement 1, wherein the single medicament provides greater than or equal to 2% sodium hypochlorite for at least one hour.

Statement 3: The method according to any one of statements 1-2, wherein the single medicament is the only irrigant needed to perform an endodontic procedure.

Statement 4: The method according to any one of statements 1-3, wherein the single medicament is used to irrigant any tooth surface, which may include a root canal, in vivo or ex vivo or in a dental lab-based setting.

Statement 5: The method according to any one of statements 1-4, wherein the single medicament is applied to the tooth surface using a 1-12mL syringe filled with the medicament attached to an appropriately sized irrigating needle between 15-32 gauge (ga), wherein the irrigating needle may be blunt cut, skived or side cut for different irrigant delivery and flow.

Statement 6: The method according to any one of statements 1-5, wherein the single medicament provides superior results to standard endodontic irrigation using NaOCl and EDTA in half the time.

Statement 7: The method according to any one of statements 1-5, wherein the single medicament provides clinically efficacious results in half the time compared to standard endodontic irrigation protocols using both NaOCl and EDTA.

Statement 8: The method according to any one of statements 1-7, wherein the calcium chelating agent is PBTC.

Statement 9: A medicament comprising:
a first component comprising at least one oxidizing agent; and,
a second component comprising at least one metal complexing agent.

Statement 10: The medicament according to statement 9, wherein the at least one oxidizing agent is sodium hypochlorite (NaOCl) and the at least one metal complexing agent is a calcium complexing agent.

Statement 11: The medicament according to any one of statements 9-10, wherein at least one of the calcium complexing agents is 2-phosphonobutane-1,2,4-tricarboxylic acid (PBTC).

Statement 12: The medicament according to any one of statements 9-11, wherein the metal complexing agent or agents provide limited stability with the at least one oxidizing agent or agents, wherein limited stability is defined as the medicament retaining greater than or equal to 2% sodium hypochlorite for at least one hour.

Statement 13: The medicament according to any one of statements 9-12, wherein at least one metal complexing agent is a phosphonate at a concentration of 2-40%.

Statement 14: The medicament according to any one of statements 9-13, wherein at least one oxidizing agent is sodium hypochlorite at a concentration of 4-12%.

Statement 15: The medicament according to any one of statements 9-14, further comprising hypochlorous acid at a concentration of 0.001%-5%.

Statement 16: The medicament according to any one of statements 9-15, wherein the ratio of HOCl to OCl—(i.e. hypochlorous acid to hypochlorite anion) is 1:20 to 1:100,000.

Statement 17: The medicament according to any one of statements 9-16, further comprising additional components that are mixed with component one and component two.

Statement 18: The medicament according to any one of statements 9-17, wherein the medicament is able to dissolve inorganic and organic debris present within smear layer.

Statement 19: The medicament according to any one of statements 9-18, further comprising at least one surfactant, builder, hydrotrope, or sequestrant.

Statement 20: The medicament according to any one of statements 9-19, further comprising a thixotropic or thickening agent.

Statement 21: The medicament according to any one of statements 9-20, wherein the at least two components are mixed at defined ratios prior to use.

Statement 22: The medicament according to any one of statements 9-21, wherein the medicament has limited stability, wherein limited stability is defined as the medicament retaining greater than or equal to 2% sodium hypochlorite for at least one hour.

Statement 23: The medicament according to any one of statements 9-22, wherein the medicament provides an antimicrobial effect.

Statement 24: The medicament according to any one of statements 9-23, wherein the medicament has a pH between 8 and 10 following the mixing of the at least two components.

Statement 25: The medicament according to any one of statements 9-24, wherein the first component has a pH between 12-13.5.

Statement 26: The medicament according to any one of statements 9-25, wherein the second component has a pH between 8-8.75.

Statement 27: The medicament according to any one of statements 9-26, wherein medicament provides at least 1.5 times more tissue dissolution than a sodium hypochlorite solution of similar concentration not containing any additives intended to boost performance.

Statement 28: The medicament according to any one of statements 9-27, wherein medicament retains its clinical effectiveness in the presence of dentin.

Statement 29: The medicament according to any one of statements 9-28, wherein the oxidizing agent comprises a hypohalogen salt.

Statement 30: The medicament according to any one of statements 9-29, wherein the calcium complexing agent comprises at least one of a phosphonate, a carboxylic acid, or a sulfonate.

Statement 31: The medicament according to any one of statements 9-30, further comprising at least one of sodium tripolyphosphate, citric acid, or a polyacrylic acid.

Statement 32: The medicament according to any one of statements 9-31, further comprising at least one an alkyldiphenyloxide disulfonate, an alcohol ethoxylate, or an amine oxide.

Statement 33: The medicament according to any one of statements 9-32, further comprising at least one of sodium xylene sulfonate, sodium lauryl sulfate, ethylhexyl sulfonate, or sodium cumine sulfonate.

Statement 34: The medicament according to any one of statements 9-33, wherein the medicament comprises a lubricant for an abrasive tool.

Statement 35: The method according to any one of statements 1-8, wherein the medicament is any one of claims 9-34.

Statement 36: A process for manufacturing each component comprising the medicament according to any one of statements 1-35 made by the process comprising:
Step 1: Dissolving any acidic ingredient(s) in water to create a mixture;
Step 2: Subsequently dissolving any builder(s) to the mixture;
Step 3: Adjusting the mixture's pH towards a neutral value using a base;
Step 4: Subsequently dissolving any surfactants or hydrotropes to the mixture;
Step 5: Adjusting the mixture's pH to a target value;

Wherein the mixture's temperature is maintained between 50 F and 140 F through the entire process.

Statement 37: The process according to statement 36, wherein the pH adjustment of step 3 targets a pH between 5-8.

Statement 38: A kit comprising the medicament of any one of claims 9-34.

Statement 39: The kit according to statement 38, wherein the medicament is provided in two separate components, wherein the first component is provided in a first container, and the second component is provided in a second container, such that the first component and the second component are not in fluid contact.

Statement 40: The kit according to any one of statements 38-39, comprising a dual-barrel syringe, wherein the first component is provided in a first barrel of the dual-barrel syringe, and the second component is provided in a second barrel of the dual-barrel syringe, such that the first component and the second component are not in fluid contact.

Statement 41: The kit according to any one of statements 38-40, further comprising any of the following: mixing vessels, mixing tips, empty syringes, application tips or brushes, irrigation tips, an instructions for use, an apex locator, an endodontic sealer, burrs, dental handpieces, gutta percha points, endodontic files, or paper points.

1. A medicament for use during root canal therapy comprising:
a first component comprising sodium hypochlorite, and
a second component comprising at least one calcium complexing agent; wherein:
the first component is mixed with the second component prior to use;
a pH of the medicament is greater than 7.5 after mixing the first and second components; and
the medicament retains greater than or equal to 2% sodium hypochlorite for at least one hour after the first and second components are mixed.

2. The medicament of claim 1, wherein the at least one calcium complexing agent comprises at least one of a phosphonate, a carboxylic acid, or a sulfonate.

3. The medicament of claim 1, wherein the at least one calcium complexing agent comprises at least one phosphonate that is 2-phosphonobutane-1,2,4-tricarboxylic acid (PBTC).

4. The medicament of claim 1, wherein the at least one calcium complexing agent comprises 2-phosphonobutane-1,2,4-tricarboxylic acid (PBTC) at a concentration of 5%-40% (w/w).

5. The medicament of claim 1, further comprising at least one of sodium tripolyphosphate, citric acid, or polyacrylic acid.

6. The medicament of claim 1, further comprising:
at least one of sodium tripolyphosphate, citric acid, or polyacrylic acid; and
at least one of an alkyldiphenyloxide disulfonate, an alcohol ethoxylate, or an amine oxide.

7. The medicament of claim 1, further comprising:
at least one of sodium tripolyphosphate, citric acid, or polyacrylic acid;
at least one of an alkyldiphenyloxide disulfonate, an alcohol ethoxylate, or an amine oxide; and
at least one of sodium xylene sulfonate, sodium lauryl sulfate, ethylhexyl sulfonate, or sodium cumine sulfonate.

8. The medicament of claim 1, wherein the sodium hypochlorite-containing component is at a pH between 12-13.5.

9. The medicament of claim 1, wherein the calcium complexing agent-containing component is at a pH between 8-8.75.

10. The medicament of claim 1, wherein the medicament is for use as the sole irrigant in performing an endodontic procedure.

11. The medicament of claim 1, wherein the first component contains sodium hypochlorite at a concentration between 4-12%.

12. The medicament of claim 1, further comprising hypochlorous acid at a concentration of 0.001%-5%.

13. The medicament of claim 1, wherein the ratio of hypochlorous acid (HOCl) to hypochlorite anion (OCl⁻) is 1:20 to 1:100,000.

14. A method of irrigating a root canal, comprising:
mixing at least a first component and a second component to yield a single medicament capable of simultaneously solubilizing organic and inorganic debris; and
administering the medicament in vivo in the root canal via a syringe and irrigating needle; wherein:
the first component comprises sodium hypochlorite; and
the second component comprises at least one calcium chelating agent,.
wherein, the single medicament comprises greater than or equal to 2% sodium hypochlorite for at least one hour after the first and second components are mixed.

15. (canceled)

16. The method according to claim 14, wherein the single medicament is the sole irrigant administered in vivo in the root canal.

17. The method according to claim 14, wherein the single medicament provides clinically efficacious results in half the time, as compared to an endodontic irrigation protocol using NaOCl and EDTA for irrigation.

18. The method according to claim 14, wherein the at least one calcium chelating agent comprises 2-phosphonobutane-1,2,4-tricarboxylic acid (PBTC) at a concentration of 5%-40% (w/w).

19. The method according to claim 14, wherein the medicament additionally provides disinfectant properties.

20. A kit comprising a medicament for root canal irrigation, wherein:
the medicament is provided as a first component and a second component, wherein the first and second components are configured to be mixed prior to applying the medicament to the root canal;
wherein the first component comprises sodium hypochlorite; and
the second component comprises at least one calcium chelating agent; and
the first component is provided in a first container and the second component is provided in a second container, such that the first component and the second component are not in fluid contact.
